(19) United States
(12) Patent Application Publication
(10) Pub. No.: US 2009/0130267 A1
(43) Pub. Date: May 21, 2009

(54) METHOD OF OPTIMIZING NUTRITIONAL VALUE OF SEEDS

(76) Inventor: John Hauck, Gloucester, MA (US)

Correspondence Address: SEYFARTH SHAW LLP, WORLD TRADE CENTER EAST, TWO SEAPORT LANE, SUITE 300, BOSTON, MA, 02210-2028, US

(21) Appl. No.: 12/122,925

(22) Filed: May 19, 2008

Related U.S. Application Data
(63) Continuation-in-part of application No. 11/598,715, filed on May 1, 2007.
(60) Provisional application No. 60/849,750, filed on Oct. 5, 2006.
(60) Provisional application No. 60/849,751, filed on Oct. 5, 2006.
(60) Provisional application No. 60/849,752, filed on Oct. 5, 2006.
(60) Provisional application No. 60/849,941, filed on Oct. 7, 2006.
(60) Provisional application No. 60/850,498, filed on Oct. 10, 2006.
(60) Provisional application No. 60/850,499, filed on Oct. 10, 2006.
(60) Provisional application No. 60/850,497, filed on Oct. 10, 2006.
(60) Provisional application No. 60/852,112, filed on Oct. 16, 2006.

(51) Int. Cl. G01N 33/02; A01C 1/00; A01C 1/06

(52) U.S. Cl. 426/231; 47/58.1 SE

(57) ABSTRACT

A method of optimizing the nutritional content of seeds to create nutritionally enriched foodstuffs for consumption by both humans and animals alike. Seeds are germinated under controlled conditions amenable to large scale, high-throughput commercial processing, and their germination is arrested at the apex of germination, where they have achieved peak nutritional value. Arrested germination seeds can be consumed directly, or dried for long term storage and later use. Additionally, nutritionally optimized seeds can be popped or puffed to create nutritionally enriched snack foods.

### CROSS REFERENCES TO RELATED APPLICATION DATA

This application is a continuation-in-part of U.S. patent application Ser. No. 11/598,715, filed on May 1, 2007, which claims priority from Provisional Patent Application No. 60/849,750, filed Oct. 5, 2006, Provisional Patent Application No. 60/849,751, filed Oct. 5, 2006, Provisional Patent Application No. 60/849,752, filed Oct. 5, 2006, Provisional Patent Application No. 60/849,941, filed Oct. 7, 2006, Provisional Patent Application No. 60/850,498, filed Oct. 10, 2006, Provisional Patent Application No. 60/850,499 filed Oct. 10, 2006, Provisional Patent Application No. 60/850,497, filed Oct. 10, 2006 and Provisional Patent Application No. 60/852,112, filed Oct. 16, 2006 all of which are incorporated herein by reference.

### FIELD OF THE INVENTION

The present disclosure relates to a method of processing seeds to increase their nutritional value, and more particularly to a method of optimizing the nutritional value of corn kernels.

### BACKGROUND OF THE INVENTION

Plant seeds generally consist of an outer seed coat, an embryo, and a storage reserve of food to sustain the embryo as it germinates. After fertilization, seeds typically enter a period of suspended development known as seed dormancy (also known as the pre-germination stage), which helps to ensure that the seed is able to survive over winter. In a dormant seed, the storage reserve generally exists in the form of oil (e.g. triacylglycerol), protein, and starch. These storage reserves are meant to withstand prolonged periods of seed dormancy, and usually consist of large, insoluble compounds. Once the period of seed dormancy is terminated (e.g. by temperature and hydration), the process of germination begins.

Seed germination is characterized by the activation of metabolic pathways designed to convert the seed's storage reserve into soluble metabolites that can fuel the growth of the embryo. For example, these metabolites may include, but are not limited to, amino acids, free fatty acids, and sugar. These metabolites are easily mobilized into the embryo, where they are used to support the growth of the embryonic plant.

An example of the conversion of the seed's storage reserve into soluble metabolites as a result of germination is detailed in Wang et al 2005 (incorporated herein by reference), which describes the metabolic profile of germination for several cultivars of peanuts. The following table, Table 2 from Wang et al 2005, shows the post-germination increase in amino acid and sucrose levels for the peanut cultivars TNS 9, TN 11, and TN 14:

TABLE 2
Changes of Sucrose, Glucose, and Total Amino Acid Contents
of Peanut Kernels before and after 9 Days of Germination

| | peanut cultivar | | |
|---|---|---|---|
| time of germination | TNS 9 | TN 11 | TN 14 |

sucrose content (mg/g freeze-dried solid)^{a,b}

| | | | |
|---|---|---|---|
| before germination | 55.9 ± 6.5^{b} | 52.2 ± 2.5^{b} | 50.3 ± 5.3^{b} |
| after germination | 99.3 ± 6.9^{a} | 88.0 ± 3.3^{a} | 116.9 ± 11.0^{a} |

glucose content (mg/g freeze-dried solid)^{a,b}

| | | | |
|---|---|---|---|
| before germination^{c} | ND | ND | ND |
| after germination | 11.8 ± 1.0 | 10.5 ± 0.8 | 11.4 ± 2.0 |

crude protein content (%, N × 5.46/defatted powder)^{a,b}

| | | | |
|---|---|---|---|
| before gemination | 37.6 ± 1.7^{a} | 37.3 ± 1.1^{a} | 38.6 ± 0.7^{a} |
| after germination | 27.1 ± 0.4^{b} | 25.6 ± 0.3^{b} | 27.9 ± 1.0^{b} |

total soluble amino acid content (mg/g protein)^{a,b}

| | | | |
|---|---|---|---|
| before germination | 0.05 ± 0.01^{b} | 0.03 ± 0.01^{b} | 0.03 ± 0.01^{b} |
| after germination | 3.22 ± 0.18^{a} | 2.26 ± 0.06^{a} | 2.58 ± 0.23^{a} |

^{a}Each value represents means ± SD (n = 3).
^{b}Data bearing different superscript letters in the same column for each cultivar and determination were significantly different (p < 0.05).
^{c}ND, not detected.

The post-germination increase in the levels of these soluble metabolites has the beneficial effect of increasing the nutritional value of the seed when it is consumed as a food source.

U.S. Pat. No. 5,738,892 (hereafter the '892 patent) describes a method of germinating cereals (e.g. husked rice, husked wheat, husked barley, husked soybean, and husked corn) to increase their nutritional content. The '892 patent uses a moisture treatment to germinate a cereal, suspends the germination, and dries the suspended-germination cereal with irradiation. A disadvantage of the '892 patent is that it is not well suited for high-throughput germination processing of cereal. Another disadvantage of the '892 patent is that the implementation of an irradiation drying scheme for the suspended-germination cereal would be cost prohibitive to install, maintain, and operate at a commercial processing scale. Yet another disadvantage is that such an irradiation system poses a health risk to workers. Still another disadvantage of this system is that a significant percentage of the world population is opposed to the irradiation of food, and will not purchase or consume food that has been subjected to this treatment.

### SUMMARY OF THE INVENTION

The present disclosure provides a method for producing seeds with an optimized nutritional content. An object of the current disclosure is to use these nutritionally optimized seeds to create nutritionally enriched foodstuffs for consumption by both humans and animals alike.

According to the disclosure, seeds are germinated to initiate the conversion of the oil, protein, and starch of their storage reserves into soluble metabolites including, but not limited to, amino acids, free fatty acids, and sugar. The metabolic conversion process that results from germination has the beneficial effect of increasing the nutritional value of the seed because the metabolic breakdown products of the storage reserves are more easily absorbed by the digestive system when these germinated seeds are consumed as food, then are the oil, protein, and starch present in the storage reserves of the pre-germinated seed.

According to one aspect of the disclosure, the germination process is allowed to continue until the seeds reach the apex of germination, which corresponds to the point where the metabolism of storage reserves has approached completion, but eruption of the root from the seed has not yet occurred. The apex of germination corresponds to the point where the seed has reached peak nutritional content. At this point, germination of the seed is arrested, because if germination is allowed to continue beyond this point, then the soluble metabolites are used to build the root and stem of the developing embryo, and the peak nutritional content of the seed starts to decline. Arrested seeds can be consumed directly. Alternatively, they can be dried for storage, at which point they can be used as, or incorporated in, foodstuffs for human consumption, or feed for animals, when needed.

It is an object of the current disclosure to create a method of optimizing the nutritional content of seeds that will benefit both humans and animals alike. The technology according to the current disclosure is likely to be of great benefit to the populations of impoverished or famine stricken countries of the world. Additionally, it is likely to be valuable technology for subsistence farmers in areas of the world afflicted with substandard growing conditions, as it will maximize the nutritional value of seed crops whose yield may be limiting in quantity. The technology according to the current disclosure is also likely to be of great utility to the agricultural industry, as it will provide a nutritionally optimized feed source for animals.

Another aspect of the current disclosure is further transforming the partially germinated, nutritionally enriched seeds into snack foods having a greater nutritional value. For example, such snack foods include, but are not limited to, popcorn, puffed wheat, puffed rice cakes, puffed trail mix, popped sunflower seeds, or puffed cereals.

It is also contemplated within the scope of the disclosure that this process can be applied to a variety of edible seeds classified as cereals (including, but not limited to, barley, fonio, kamut, corn, pearl millet, oats, palmer's grass, rice, rye, sorghum, spelt, teff, triticale, wheat, and wild rice), pseudocereals (including, but not limited to, breadnut, buckwheat, cattail, chia, cockscomb, grain amaranth, kañiwa, pitseed goosefoot, quinoa, acacia seed), legumes (including, but not limited to, bambara groundnut, chickpeas, cowpeas, fava beans, hyacinth beans, lablab, lentils, lupins, peas, peanuts, pigeon peas, velvet beans, vetch, winged beans, yam beans, tonka beans, and soybeans), nuts (including, but not limited to, almond, beech, butternut, brazil nut, candlenut, cashew, chestnuts, colocynth, filbert, pecan, shagbark hickory, kola nut, macadamia, mamoncillo, maya nut, mongongo, acorns, ogbono nut, paradise nut, pili nut, walnut, and water caltrop), nut-like gymnosperm seeds (including, but not limited to, cycads, gingko, juniper, monkey puzzle, pine nuts, and podocarps), and a variety of miscellaneous edible seeds (including, but not limited to, cempedak, egusi, fox nut, fluted pumpkin, hemp seed, jackfruit, lotus, malabar gourd, pistachio, pumpkin, and sunflower). It is also contemplated within the scope of the disclosure that this process can be applied to a variety of edible vegetable seeds and fruit seeds.

It is also contemplated within the scope of the disclosure that varying the degree of germination during this process can be used to vary the resulting nutritional level of the seed.

Without being bound to any particular theory, it is also contemplated within the scope of the disclosure that the germination process may produce seeds that have novel properties that are of utility in the foodstuffs industry, as well as in other industries. For example, germination of corn kernels may result in the ability to produce a corn mash for distillation that yields spirits with unique flavors and qualities. Similarly, germination of corn kernels may result in the ability to enhance the production of ethanol from the resulting corn mash. As another example, germination of peanuts is correlated with an increase in the production of resveratrol, a potent phytochemical thought to function as an anti-cancer agent, and germinated peanuts are believed to be able to produce biomedically relevant products.

Another object of the current disclosure is to provide a method of increasing the nutritional content of seeds in a manner that is high-throughput and scalable for commercial production. According to the disclosure, it is possible to accelerate the germination time of seeds by exposing them to higher temperatures. For example, this could be accomplished by immersing them in temperature controlled water baths with elevated temperatures. Such temperature controlled water baths are already implemented in a wide variety of commercial food production/processing facilities (e.g. chilling tanks in poultry processing). This has the beneficial effect of allowing seed processing facilities to be developed with standardized equipment, which increases food safety and also lowers production costs.

Another aspect of the current disclosure is to provide a method of estimating the apex of germination for a batch of germinating seeds en masse. According to the disclosure, it is possible to determine a seed germination protocol, and then process a number of test batches of seeds by this seed germination protocol under identical conditions. Each test batch is then monitored until a certain percentage of the test batch shows root eruption, and then the germination of the batch is arrested. For example, test batch 1 is arrested when about 2% of the seeds show root eruption, test batch 2 is arrested when about 5% of the seeds show root eruption, test batch 3 is arrested when about 10% of the seeds show root eruption, and test batch 4 is arrested when about 15% of the seeds show root eruption. Each batch is then analyzed to determine the progress of germination through all seeds in the batch. The percentage of monitored root development that corresponds to the batch with the highest percentage of seeds within the population that are at the apex of germination is then used as the pre-determined percentage of root eruption to estimate the apex of germination when seed processing is scaled up for mass production. This type of developmental analysis is expensive, and this method reduces production costs by limiting the expense of such analysis to a small number of test batches at the initial stage of production. This also has the beneficial effect of further increasing the throughput capacity of seed processing, as well as the processing efficiency.

It is yet another aspect of this disclosure to provide a low-cost, effective, safe, and high-throughput method of drying the arrested-germination seeds based on existing convection oven technology.

### BRIEF DESCRIPTION OF THE DRAWINGS

The abovementioned and other features and advantages of the present disclosure will be better understood when reading the following detailed description of an illustrative embodiment, taken together with the following drawings in which:

FIG. 1 depicts a process diagram for optimizing the nutritional content of corn kernels.

### DETAILED DESCRIPTION OF THE INVENTION

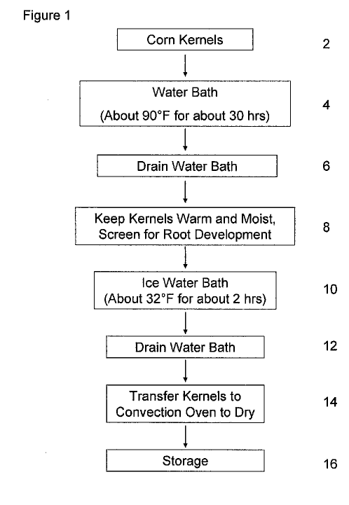

According to the current disclosure, the nutritional value of corn kernels can be optimized as shown in the process flow diagram of FIG. 1. Corn kernels (2) are immersed in a temperature controlled water bath (4) to initiate the process of seed germination. In this illustrative embodiment, corn kernels are immersed in a water bath (4) at a temperature of about 90° F. for about 30 hours in order to accelerate the germination process. It is also contemplated within the scope of the disclosure that germination could be initiated in a variety of different fluids, for example ethanol or an aqueous solution. It is also contemplated within the scope of the disclosure that germination could be initiated in a variety of different mediums, for example a nitrogen atmosphere or moist sand.

A variety of methods are available for breaking seed dormancy and initiating seed germination, and the current disclosure is not limited to using water. For example, another embodiment of the current disclosure could expose seeds to Gibberellic Acid in order to initiate germination. One skilled in the art will recognize that the method best employed for initiating seed germination will vary depending on the type of seed being processed and the purpose for which the processed seed is to be used.

Different temperature and time combinations may be used in order to facilitate the production process of corn kernels (2) in a large scale implementation of the current disclosure.

Once the germination process has been initiated, the temperature controlled water bath (4) is drained (6), and the corn kernels (2) are transferred to a warm, moist environment (8) in which they can easily be monitored for root development.

Corn kernels (2) are allowed to develop to a point where they have reached the apex of germination, which correlates to an optimal, peak nutritional value. Without being bound to any particular theory, the apex of germination (and also the resulting optimal, peak nutritional value) is considered to be the point at which the metabolism of the storage reserves of a given corn kernel has approached completion, but eruption of the root from the seed has not yet occurred. If the seed is allowed to germinate past the apex of germination, the resulting nutritional value decreases progressively from the peak nutritional value for as long as germination is allowed to continue.

One aspect of the current disclosure is to provide a method of estimating the apex of germination for a population of corn kernels (2) that are being processed according to the disclosure. One skilled in the art will recognize that it is difficult to completely synchronize the germination of a large population of corn kernels (2). According to the current disclosure, the germination process for a population of corn kernels (2) is considered to be a continuum of development across the population as a whole. As the corn kernels (2) are incubated in a warm, moist environment (8) they are monitored for corn kernels (2) that show root eruption. Once root eruption is detected for a pre-determined percentage (e.g. about 5% or about 10%) of the population of corn kernels (2), germination of the population is arrested by transferring the corn kernels (2) to an ice water bath (10).

According to the disclosure, the pre-determined percentage of the population of corn kernels (2) to be used to estimate peak nutritional value of the corn kernel (2) population can be determined empirically. For example, individual small scale test batches of corn kernels can be processed under identical conditions and monitored for different percentages of root eruption (e.g. about 1%, 2%, 3%, 5%, 10%, etc.), and then analyzed for progression of germination within the sample to determine which percentage of root eruption maximizes the number of corn kernels that have reached the apex of germination within the batch as a whole. In other words, the percentage of monitored root development that corresponds to the batch with the highest percentage of seeds within the population that are at the apex of germination is then used as the pre-determined percentage of root eruption to estimate the apex of germination when seed processing is scaled up for mass production.

One skilled in the art will recognize that this percentage may vary for a number of additional reasons (e.g. climate, season, ambient temperature, humidity, etc.). As an example, corn kernel processing facilities located in different geographical regions may screen for different percentages of root eruption to estimate the apex of germination of their processed batches of corn kernels (2), even when using germination protocols that are otherwise identical. Additionally, the ideal percentage will likely vary for different seed species and different seed processing applications, but in general it is thought to range between about 1% and about 33%.

One skilled in the art will also recognize that seed monitoring can occur by a number of different methods. For example, seeds could be monitored visually, or the monitoring could be automated using a variety of electro-optical systems. It is also contemplated within the scope of the disclosure that root eruption is correlated with changes in a variety of physical properties of the seeds that may also be amenable to monitoring. For example, progression of germination and root eruption may result in a variety of seed changes including, but not limited to, changes in seed opacity, seed temperature, seed mass, seed density, or seed buoyancy.

In one illustrative embodiment, the ice water bath (10) is maintained at about 32° F. and the corn kernels (2) are incubated for about 2 hours to completely arrest the germination process. Other combinations of temperature and time may be implemented to stop germination, and these combinations can be empirically determined for specific seed types, or specific seed processing applications.

Once germination has been arrested, the ice water bath (10) is drained, and the corn kernels (2) are removed. In one embodiment, the nutritionally optimized corn kernels can be consumed immediately. In one alternative embodiment, the corn kernels can be dried in a standard convection oven (14) to desiccate the nutritionally enriched corn kernels (2) for long term storage (16). Nutritionally enriched corn kernels may be used in creating foodstuffs for consumption by both humans and animals. For example, they can be popped to create a nutritionally enriched snack food in the form of popcorn. As another example, they can be sliced or cracked to produce a nutritionally enriched feed for cows and chickens. In yet another example, they can be ground to create a nutritionally enriched meal for use in baked goods including, but not limited to, bread, chips, or tortillas.

The above detailed description presents one illustrative embodiment of the current disclosure in the form of a method for optimizing the nutritional value of corn kernels. For the sake of clarity, terminology is used in this detailed description that is specific for this particular embodiment of the disclosure; however, this terminology is not intended to be limiting, and should not be construed as limiting insofar as one skilled in the art will recognize that many different forms and variations of the current disclosure are possible within the scope of the appended claims.

What is claimed is:

1. A method of optimizing the nutritional content of seeds, comprising:
initiating germination of said seeds;
assessing germination state of said seeds; and
arresting said germination of said seeds at the apex of germination.

2. The method of claim 1, wherein said seeds are placed in a humidity controlled environment to initiate germination.

3. The method of claim 2, wherein the temperature of said temperature controlled bath is between about 70° F. and about 90° F.

4. The method of claim 1, wherein said seeds are exposed to a Gibberellic Acid to initiate germination.

5. The method of claim 1, wherein said germination state of said seeds is assessed by monitoring and selected from the group consisting of visual monitoring, electro-optical monitoring, size monitoring, opacity monitoring, temperature monitoring, mass monitoring, buoyancy monitoring, and density monitoring.

6. The method of claim 1, wherein said apex of germination is correlated with root development for a predetermined percentage of said seeds.

7. The method of claim 6, wherein said predetermined percentage is between about 1% and about 50% of the total of said seeds.

8. The method of claim 1, wherein said germination of said seeds is arrested by placing said seeds in a low temperature environment.

9. The method of claim 1, wherein said seeds arrested at said apex of germination are desiccated for storage.

10. The method of claim 9, wherein said seeds are desiccated in a convection oven.

11. The method of claim 1, wherein said seeds are derived from the groups consisting of legumes, cereal, pseudo-cereal, nuts, nut-like gymnosperms, gourd seeds, vegetable seeds, fruit seeds, and flower seeds.

12. The method of claim 11, wherein said cereal is derived from the group consisting of corn, barley, fonio, kamut, pearl millet, oats, palmer's grass, rice, rye, sorghum, spelt, teff, triticale, wheat, and wild rice.

13. The method of claim 12, wherein said desiccated corn is popped to create a nutritionally enriched snack food.

14. The method of claim 12, wherein said desiccated corn is processed into a nutritionally enriched meal.

15. The method of claim 12, wherein said corn is derived from the strain Crow's Hybrid, High Lysine SR218.

16. A method of estimating the nutritional value of a population of germinating seeds, comprising:
determining a seed germination protocol for germinating said seeds;
processing multiple test batches of said seeds via said seed germination protocol;
screening said multiple test batches individually for different percentages of said seeds showing root eruption;
subjecting said screened multiple test batches to analysis to determine the number of said seeds that have reached the apex of germination in each of said screened multiple test batches as a whole;
determining empirically said percentage of said seeds showing said root eruption that correlates with the highest number of said seeds at said apex of germination in said individually screened test batch as a whole; and
using said empirically determined percentage to estimate said apex of germination of said population of germinating seeds.

17. A method of producing nutritionally enriched foodstuffs, comprising:
initiating germination of corn kernels;
assessing germination state of said corn kernels;
arresting germination of said corn kernels at the apex of germination; and
popping said arrested corn kernels to create a nutritionally enriched snack food.

18. The method according to claim 17 wherein said arrested corn kernels are dried prior to being popped.
